(19) United States
(12) Patent Application Publication
(10) Pub. No.: US 2007/0173693 A1
(43) Pub. Date: Jul. 26, 2007

(54) Endoscope

(75) Inventor: Moshe Refael, Nofit (IL)

Correspondence Address: Mark M Friedman;Bill Polkinghorn, 9003 Florin Way, Upper Marlboro, MD, 20772, US

(73) Assignee: IMAGE IN LTD., Haifa (IL)

(21) Appl. No.: 10/571,395

(22) PCT Filed:
(86) PCT No.: PCT/IL04/00819

Related U.S. Application Data
(60) Provisional application No. 60/500,990, filed on Sep. 9, 2003.

(51) Int. Cl. A61B 1/00

(52) U.S. Cl. 600/144; 600/152; 600/139; 600/146

(57) ABSTRACT

An endoscope (1000) comprising a flexible elongated solid body (110) comprising cavities wherein at the distal portion of the body there is a section with greater flexibility than other portions of the body, for enabling an operator to steer the head (200) along twists and convolutions and a stiffening mechanism (480) for hardening at least a portion of the body.

### FIELD OF THE INVENTION

The invention relates to an intra-corpus imaging instrument.

### BACKGROUND TO THE INVENTION

Endoscopes are used for a variety of examinations and treatments in lumens in the body. For example, colonoscopes and gastroscopes for the digestive system, bronchoscopes for the respiratory system, etc.

Endoscope units often comprise two parts: an inserted part, which penetrates into the body lumen, and a handle, which is used by the doctor to hold and control the unit.

The inserted part comprises a long, flexible trunk coming out of the handle, followed by gimbals topped by the head. The gimbals enable the head to turn in any direction for steering the unit. The head comprises a vision system as well as various ingresses and egresses for operating the unit and for operating various medical systems via the unit. For example, the head can include lenses for viewing, light sources for illuminating the field of view, and apertures for delivering various materials and instruments, such as liquids to clean the lens, liquids to distend the lumen, fluoroscopes, sampling devices, and therapeutic devices. The inserted part comprises several thin canals leading to the apertures, as well as a connection to the lenses and a power connection for the light sources. The connection to the lenses can comprise a fiber optic cable to carry the image or, if the lens is provided with CCD circuitry to convert the image to data, a data link.

The unit also houses steering cords connected to controls on the handle that enable a user to steer the endoscope by retracting or extending the cords. The steering cords run to the head of the endoscope, where the gimbals enables the head to be steered in any direction, thereby enabling the doctor to steer the unit.

Insertion and retraction of the unit is usually done manually by the doctor who physically pushes the trunk into the body lumen, steering by means of the handle.

Ancillary equipment connected to the proximal end of the endoscope unit are collectively referred to as a workstation and can include:

1. Water pump system.

2. Compressor for air pressure.

3. Electrical power supply for light.

4. Image processing unit.

5. Vacuum pump.

The ancillary equipment connects to the endoscope via tubes and cables. Therefore an improved endoscope can be connected to an existing workstation by use of an adaptor that interfaces between the workstation connections and the endoscope connections.

Existing endoscopes are built for repeated use and therefore must be cleaned and disinfected between uses. This is an expensive time-consuming task. As a corollary, to ensure that a clean endoscope is available at all times, a stock of endoscopes must be maintained, since at any given time some of the endoscopes may not be ready for reuse. It would be preferable to provide a disposable, sterile endoscope. Existing endoscopes are too expensive to be disposable.

Existing endoscopes also have a high amortization rate caused by bending that breaks interior parts of the endoscope, by chemicals used in the cleaning of the system that attack the lenses, and by liquids that eventually infiltrate the electronic components of the device. Endoscope repair is typically very expensive. On average, an endoscope goes out of service within a thousand uses.

The present invention provides an economical mass produced design, enabling the endoscope to be disposable. The trunk, gimbals, and head are all mass-produced, for example with extruded plastic.

In one embodiment, the gimbals comprise a series of links interconnected by cardan joints, perforated for passage of internal tubes and cables and connected to one another by two opposed thin hinges (cardan joint), with the hinges on every other pair of disks situated at 90 degrees to the hinges of the previous disk pair. In this configuration, navigational control wires running from the handle to the head of the device can be pulled and released to steer the head, the gimbals providing freedom of movement in any direction.

In an alternative embodiment, the gimbals consist of a simple convoluted tube comprising thicker inner rings alternating with thinner outer rings, wherein the outer rings provide the required freedom of movement.

The typical endoscope is a complex metal device comprising a housing in which each internal cable and tube running from the workstation to the head has its own casing. The need for individual casings is an added expense. A further advantage of using an extruded solid flexible tube for the trunk is that it comprises lumens for the internal cables and tubes, thereby eliminating the need for casings for each individual cable and tube.

As the endoscope is inserted into the patient, it is desirable to have a means for varying the stiffness of the trunk. For example, when the trunk is starting to go through a sharp curve, it is desirable to increase stiffness (sacrificing flexibility) in order to prevent the trunk from buckling from opposing forces generated by the insertion force on the one hand and the resistance on the head on the other hand.

It is therefore desirable to have a user-controlled stiffening element for the trunk. The stiffening element could be a cable pulling on the steering cables thereby compressing the trunk, additional separated cable or cables, wires inserted into the trunk to directly impart stiffness, or a fluid (liquid or gas) inserted into the trunk to directly impart stiffness.

Manual insertion of the endoscope is not always the best method since it can accidentally cause internal punctures in the body. It is therefore desirable to have alternative means of driving the head. There are several possibilities, including pumping a stream of liquid against the inside of the head or mechanically imparting linear motion on the trunk, for example by rollers or other auxiliary propelling device.

Another problem associated with endoscopes is providing maximum size and number of access channels in the tube. One of the limiting factors is the geometries of the channel cross sections, particularly at the distal end of the endoscope), which are normally circular in shape. A better use of space can be achieved by varying the cross-sectional shape of these elements according to the needs of the particular application. For example, if a larger diameter tool channel is required, the cleaning water channel can be made crescent-shaped to accommodate the area needed for the tool channel. In other words, instead of just circular, the cross sectional shape of the channel can be other geometrical or any other shape—referred to herein as polymorphic.

Another problem associated with endoscopes is that the viewing lens must be kept a minimum distance from the wall of the lumen to avoid occlusion of the lens. This can be prevented by covering the lens with a cover, which may be a cap incorporated with the lens assembly or a separate cap.

Another problem associated with endoscopes is that the intensity of the light sources used for illuminating the device's viewing field and in the fluoroscope is fixed. This can be improved with the addition of an adjustable power control, such as a rheostat to the light sources.

Accordingly, several objects and advantages of preferred embodiments of the present endoscope invention are (some features are optional):
low cost disposable and sterile
mass produced from an inexpensive material
head, gimbals, and/or trunk are produced cheaply enough to justify making the unit disposable.
head, gimbals, and/or trunk are produced employing injection molding or extrusion or other manufacturing method.
head, gimbals, and/or trunk are connected together with simple pressure-fit connector or manufactured as one piece
propelled by manual insertion, liquid pressure, or drive wheel
user-controlled trunk stiffness (complete or partial) by tensioning steering cords, inserting reinforcing fluid, or inserting reinforcing wires
cross-sectional shapes and areas of the channels running through the trunk and their outlets in the head are adjusted as necessary to achieve the most efficient footprint.
lenses are protected by a cap
the intensity of the light sources is user-controlled
CCD (or CMOS or other imaging device) is placed substantially perpendicular to or inclined with respect to the line of sight so that its side faces the head, and a mirror under the lens redirects the viewed image onto the CCD at the correct angle, thereby maintaining minimum CCD footprint.
the drive-control handle is reusable
an adapter connects the endoscope to a workstation (which may be of any existing make).

### BRIEF DESCRIPTION OF THE INVENTION

There is thus provided, in accordance with some preferred embodiments of the present invention, an endoscope for use in connection and combination with controls and a workstation, the endoscope for inserting into a body lumen to diagnose or treat a medical condition, the endoscope comprising:

flexible elongated solid body having a proximal portion and a distal portion with a head, the body comprising cavities provided for passing to or from the head materials, data connections, power connections, or instruments, wherein at the distal portion of the body there is a section with greater flexibility than other portions of the body, for enabling an operator to steer the head along twists and convolutions in the lumen; and

a stiffening mechanism for hardening at least a portion of the body, so as to allow hardening of said at least a portion of the body during navigation of the endoscope within the body lumen.

Furthermore, in accordance with some preferred embodiments of the present invention, the stiffening mechanism comprises one or more cables, connected to the head or other parts of the body, which when pulled effectively stiffen said at least a portion of the endoscope.

Furthermore, in accordance with some preferred embodiments of the present invention, the stiffening mechanism comprises at least one elongated insert for insertion into a cavity and provide added stiffness to said at least a portion of the endoscope.

Furthermore, in accordance with some preferred embodiments of the present invention, the stiffening mechanism comprises at least one cavity for filling with a fluid.

Furthermore, in accordance with some preferred embodiments of the present invention, the fluid comprises liquid.

Furthermore, in accordance with some preferred embodiments of the present invention, the fluid comprises gas.

Furthermore, in accordance with some preferred embodiments of the present invention, the endoscope is further provided with a reservoir for containing the fluid and providing it when desired.

Furthermore, in accordance with some preferred embodiments of the present invention, the cross-sections of at least some of the cavities are circular.

Furthermore, in accordance with some preferred embodiments of the present invention, the cross-sections of at least some of the cavities are polygonal.

Furthermore, in accordance with some preferred embodiments of the present invention, the cross-sections of at least some of the cavities are amorphous.

Furthermore, in accordance with some preferred embodiments of the present invention, the cross-sections of at least some of the cavities are characterized as lacking symmetry.

Furthermore, in accordance with some preferred embodiments of the present invention, the section with greater flexibility comprises a plurality of links interconnected by cardan joints.

Furthermore, in accordance with some preferred embodiments of the present invention, the section with greater flexibility comprises a concatenated section.

Furthermore, in accordance with some preferred embodiments of the present invention, the endoscope is further provided with an auxiliary propelling unit for coupling with and propelling the endoscope.

Furthermore, in accordance with some preferred embodiments of the present invention, the auxiliary propelling unit is operable manually.

Furthermore, in accordance with some preferred embodiments of the present invention, the auxiliary propelling unit is motor-operated.

Furthermore, in accordance with some preferred embodiments of the present invention, at least some of the cavities with outlets at external surfaces of the distal portion facilitating exit of jets of fluid from the outlets thus providing jet propulsion to the endoscope.

Furthermore, in accordance with some preferred embodiments of the present invention, the body is made of several separate parts, that are connectable.

Furthermore, in accordance with some preferred embodiments of the present invention, the parts are provided with snap connectors for fast connection.

Furthermore, in accordance with some preferred embodiments of the present invention, cords are further provided, inserted through at least some of the cavities, each cord connected to the head, thus enabling manipulating orientation of the head by pulling one or more cords.

Furthermore, in accordance with some preferred embodiments of the present invention, there is provided an endoscope for use in connection and combination with controls and a workstation, the endoscope for inserting into a body lumen to diagnose or treat a medical condition, the endoscope comprising:

flexible elongated solid body having a proximal portion and a distal portion with a head, the body comprising cavities provided for passing to or from the head materials, data connections, power connections, or instruments, wherein at the distal portion of the body there is a section with greater flexibility than other portions of the body, for enabling an operator to steer the head along twists and convolutions in the lumen; and

an orientation control mechanism for controlling the orientation of the head, comprising at least one of a plurality of cords passing through at least one of the cavities and connected to the distal portion, allowing manipulating of the orientation of the head by pulling or releasing some or all of the cords.

Furthermore, in accordance with some preferred embodiments of the present invention, there is provided an endoscope for use in connection and combination with controls and a workstation, the endoscope for inserting into a body lumen to diagnose or treat a medical condition, the endoscope comprising:

flexible elongated solid body having a proximal portion and a distal portion with a head, the body comprising a solid body provided with a plurality of cavities for passing to or from the head materials, data connections, power connections, or instruments, wherein at the distal portion of the body there is a section with greater flexibility than other portions of the body, for enabling an operator to steer the head along twists and convolutions in the lumen.

Furthermore, in accordance with some preferred embodiments of the present invention, there is provided an endoscope for use in connection and combination with controls and a workstation, the endoscope for inserting into a body lumen to diagnose or treat a medical condition, the endoscope comprising:

flexible elongated solid body having a proximal portion and a distal portion with a head, the body comprising cavities provided for passing to or from the head materials, data connections, power connections, or instruments, wherein at the distal portion of the body there is a section with greater flexibility than other portions of the body, for enabling an operator to steer the head along twists and convolutions in the lumen; and

an auxiliary propelling device, adapted to be engaged to the flexible elongated solid body, and help in advancing and maneuvering the endoscope in the body lumen.

Furthermore, in accordance with some preferred embodiments of the present invention, the auxiliary propelling device comprises a mechanical mechanism operable manually.

Furthermore, in accordance with some preferred embodiments of the present invention, the auxiliary propelling device comprises a motor-operated mechanical mechanism.

Furthermore, in accordance with some preferred embodiments of the present invention, there is provided an endoscope for use in connection and combination with controls and a workstation, the endoscope for inserting into a body lumen to diagnose or treat a medical condition, the endoscope comprising:

flexible elongated solid body having a proximal portion and a distal portion with a head, the body comprising cavities provided for passing to or from the head materials, data connections, power connections, or instruments, wherein at the distal portion of the body there is a section with greater flexibility than other portions of the body, for enabling an operator to steer the head along twists and convolutions in the lumen, further provided with cavities with outlets located externally on the flexible elongated solid body, so as to facilitate jets of fluid, when such fluid is pressurized into the cavities, for jet propulsion of the endoscope.

Furthermore, in accordance with some preferred embodiments of the present invention, there is provided a method for manufacturing an endoscope having a flexible elongated solid body, the body having a proximal portion and a distal portion with a head, the body comprising cavities in the form of a plurality of channels running through it and provided for passing to or from the head materials, data connections, power connections, or instruments, comprising manufacturing the elongated body in extrusion.

Furthermore, in accordance with some preferred embodiments of the present invention, the method further comprises manufacturing the head employing casting.

### BRIEF DESCRIPTION OF THE FIGURES

FIG. 1 illustrates an endoscope in accordance with a preferred embodiment of the present invention.

FIG. 2A illustrates in section view, a connector component for connecting primary components of an endoscope in accordance with a preferred embodiment of the present invention.

FIG. 2B illustrates in greater detail the connector of FIG. 2A.

FIG. 3 illustrates in side section view the viewing components in the head of an endoscope in accordance with a preferred embodiment of the present invention.

FIG. 4 illustrates in top view the distal end of an endoscope in accordance with a preferred embodiment of the present invention.

FIG. 5A illustrates in isometric view, steering controls and stiffening controls of an endoscope in accordance with a preferred embodiment of the present invention.

FIG. 5B illustrates in side section view, components for stiffening the endoscope trunk by tensioning of the steering cords in accordance with a preferred embodiment of the present invention.

FIG. 6 illustrates in side section view, components for stiffening the endoscope trunk by pumping in fluids in accordance with an alternative preferred embodiment of the present invention.

FIG. 7 illustrates in side section view, components for stiffening the endoscope trunk by insertion of reinforcing cables in accordance with an alternative preferred embodiment of the present invention.

FIG. 8 illustrates in side section view a fluid-propulsion mechanism for an endoscope in accordance with a preferred embodiment of the present invention.

FIG. 9A illustrates in side view a mechanical propulsion mechanism for an endoscope in accordance with an alternative preferred embodiment of the present invention.

FIG. 9B illustrates the propulsion mechanism of FIG. 8A in isometric view.

FIG. 10A illustrates in isometric view gimbals (with the outer shell removed) of an endoscope in accordance with a preferred embodiment of the present invention.

FIG. 10B illustrates in detail the section shown in FIG. 9A.

FIG. 10C illustrates in section view, the gimbals of FIGS. 9A and 9C.

FIG. 11 illustrates in isometric view alternative gimbals in accordance with an alternative preferred embodiment of the present invention.

FIG. 12A illustrates in top section, the trunk of an endoscope in accordance with a preferred embodiment of the present invention.

FIG. 12B illustrates the two side section views indicated in FIG. 12A.

### DETAILED DESCRIPTION OF THE INVENTION

The present invention is an endoscope for working inside lumens of a patient's body. The proximal end of the endoscope is normally connected to a workstation, which can comprise various ancillary devices, such as water pump, air compressor, electrical power supply etc.

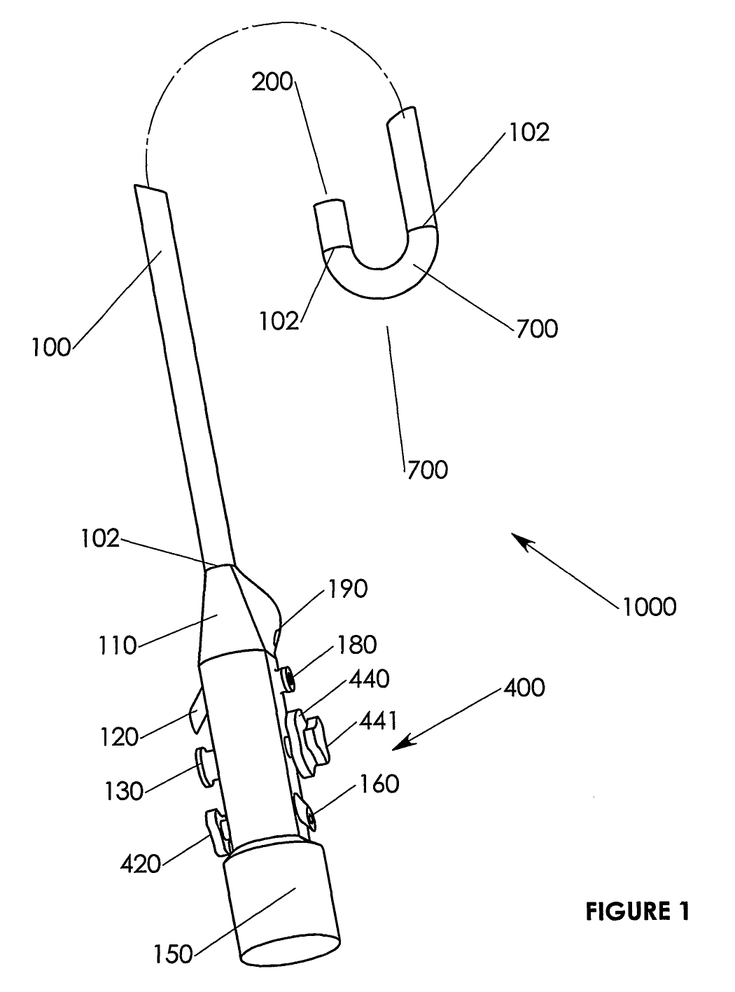

FIG. 1 is a general view of the endoscope 1000 of the present invention with trunk 100 having a distal end (or head) 200 and proximal end 150. The length of trunk 100 depends on the desired maximum insertion distance, typically several meters. In FIG. 1, omitted portions of trunk 100 are indicated by a dashed curved line. Head 200 is inserted into a patient's lumen and comprises components for viewing and openings for injecting and withdrawing materials and devices. Head 200 can be steered by an operator by various means as are known in the art. In a preferred embodiment of the present invention, steering is accomplished by retracting and releasing cables passing through trunk 100 and gimbals 700 and attached to head 200. Head 200 is mounted on gimbals 700, which enables it to be steered freely in any direction. Trunk 100 is flexible enough to follow head 200 and gimbals 700.

In a preferred embodiment of the present invention shown in FIG. 1, proximal end 150 is part of a handle 400 with controls whereby an operator controls steering of and insertion/removal of head 200 into/from the patient's lumen and whereby the operator inserts and removes materials, connections, and instruments (such as a fluoroscope) to/from channels running through trunk 100 and gimbals 700 to head 200. The materials can be various states of matter (e.g., gas, liquid, solid); the connections can be various types including power supply for a viewing light or data link for receiving viewing data; the instruments can include sampling probes, fluoroscopes, therapeutic devices, and other medical instruments. It will be recognized by one skilled in the art that the user-control interface provided by handle 150 can be implemented in various ways without affecting the primary innovations of the present invention. Thus, for example, the controls could be implemented on a stationary box, rather than a handle. Handle 400 is optional.

The precise use, location, and existence of these controls and ingress points is not critical to the novelty and innovation of the current patent. They are provided for reference, and can include:
Ingress 120 for power for navigation light source, for fluoroscope light source, and for other electrical components.
Egress 130 for vacuuming out liquids
Optional interface to existing workstation 150
Ingress 160 for fluids for cleaning lens at distal end
Conduit 180 for data connections
Conduit 190 for inserting diagnostic and therapeutic instruments.
Control 420 for governing stiffness of trunk 100
Steering controls 440 and 441

In a preferred embodiment of the present invention, the power 120 for the navigation light source and/or the fluoroscope light source can be equipped with a rheostat, so that the operator can control the intensity of the light.

In a preferred embodiment of the present invention, several components of the invention are separate pieces connected 102 together. These separate pieces include handle 400, trunk 100, gimbals 700, and head 200. Alternatively, all or some of these pieces can be manufactured as a single unit.

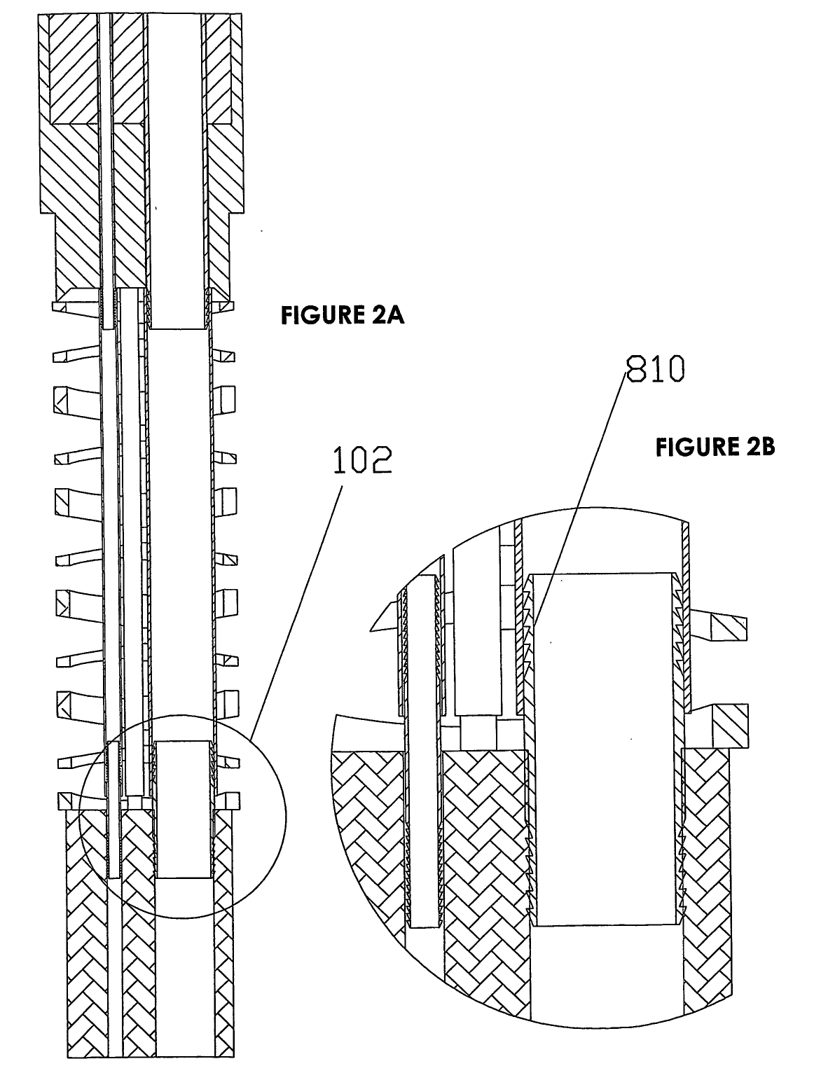

FIG. 2A and FIG. 2B illustrate a connection point 102 with a connector 810 that can be used for fast, one-time connection of the pieces. Connector 810 comprises a barbed plug inserted into the adjacent ends of each piece.

One skilled in the art will realize that there are many other types of connectors that can be used to connect the parts. For example, in an alternative preferred embodiment of the present invention, the handle is reusable while the trunk, gimbals and head are disposable. In that embodiment, a reusable lock ring can be used to connect the handle to the trunk, while barbed one-time plugs connect the trunk to the gimbals and the gimbals to the head.

The present invention can be manufactured using inexpensive mass production, for example extruded plastic, in which case it can affordably used as a one-time, disposable device. Alternatively, it can be adapted to include both disposable and reusable pieces. For example, handle 400 could be reusable and connected to disposable trunk 100, gimbals 700, and head 200.

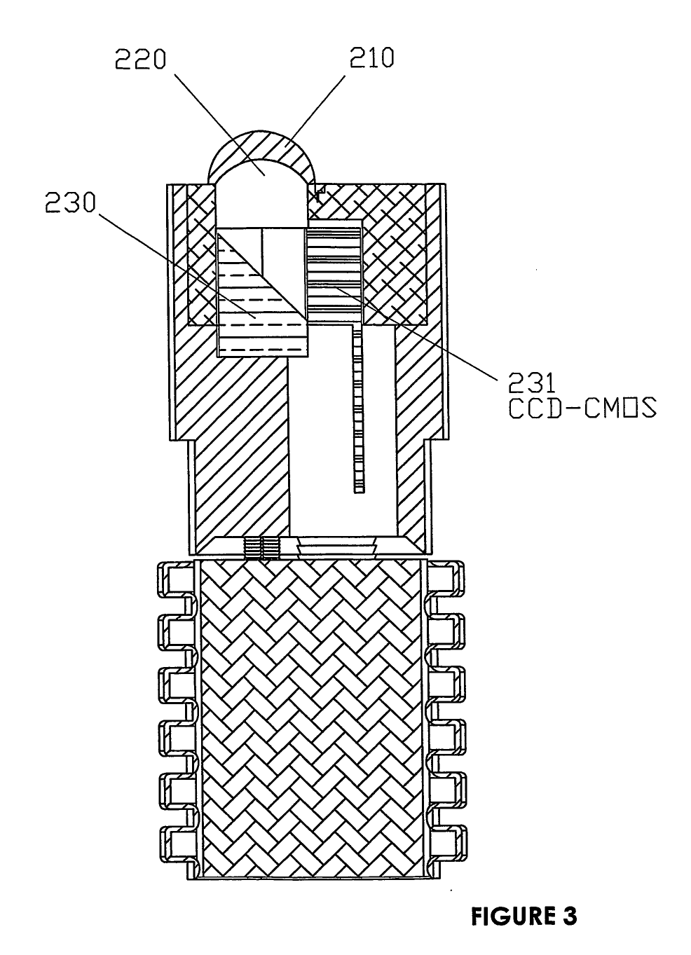

FIG. 3 illustrates in side section view, the viewing components in the head 200 of an endoscope 1000 in accordance with a preferred embodiment of the present invention.

An innovation of the present invention is provided by protective cap 210 covers viewing lens 220, with a gap between the two. Protective cap 210 ensures that if lens 220 is pushed into contact with another object, such as the wall of the lumen, that the gap between cap 220 and lenses 210 maintains depth of field, keeping the image in focus and preventing occlusion by the object. Protective cap 210 is made of translucent, durable material, such as plastic.

Another innovation of the present invention is provided by a light-bending component 230, such as a mirror or prism, which reflects images received through lens 220 at a 90 degree angle to CCD component 231, thereby enabling CCD 231 and its related electronics to be installed on its side, such that CCD's 231 longest dimension lies in parallel with the body of trunk 100, thereby keeping the area of trunk 100 cross-section taken up by CCD 231 to a minimum, thereby leaving the most space possible for other components such as the channels for inserting and removing materials and instruments.

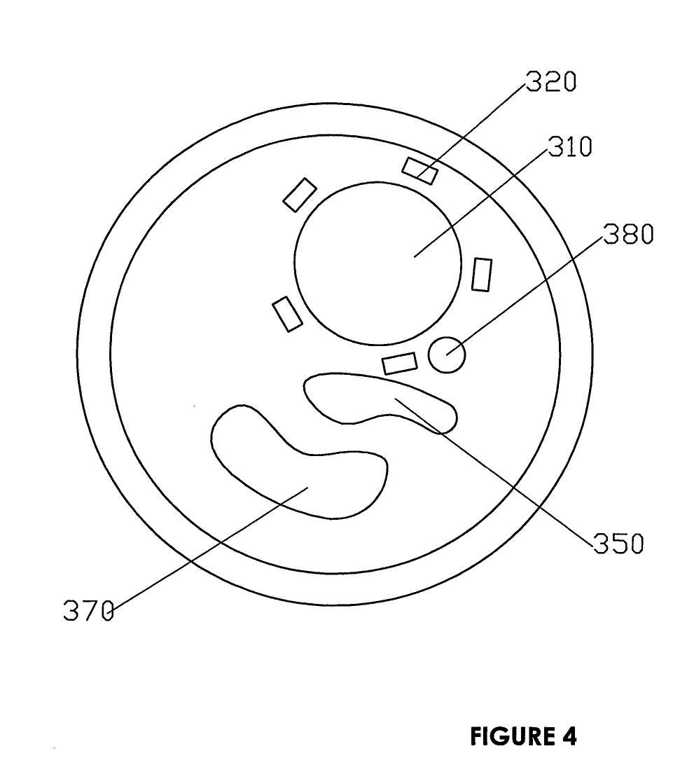

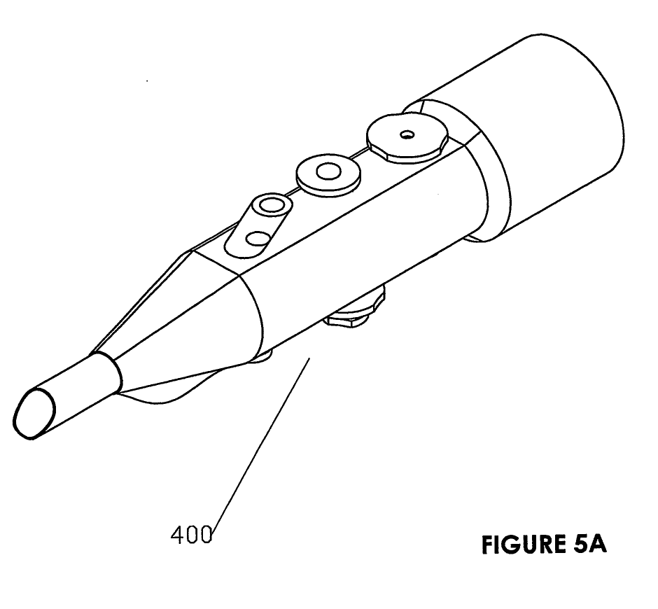

FIG. 4 is a top view of head 200. As shown in the figure, another innovation of the present invention is to vary the cross-sectional shapes and areas of the internal channels 370 used to insert/remove materials/instruments to/from head 200 and of fluoroscope channel 350. Both these channels are shaped for most efficient use of space. For example, if channel 370 were the traditional round shape, it would take up more area of the cross section, thereby limiting the cross-sectional area of channel 350 and of other channels.

The figure shows lens cap 310 (same as 210 in FIG. 2A), light sources 320 (for example, light emitting diodes—LEDs), and lens cap cleanser dispenser 380. In a preferred embodiment of the present invention, light sources 320 and fluoroscope 350 are equipped with rheostats at handle 400 so that the operator can control the light intensity.

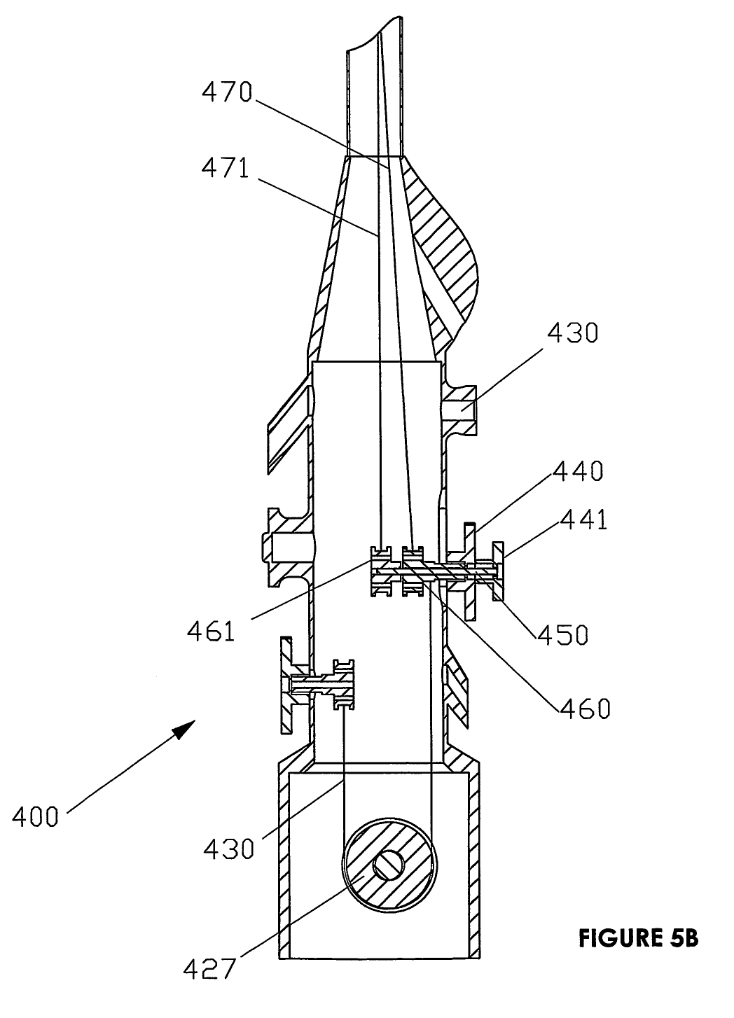

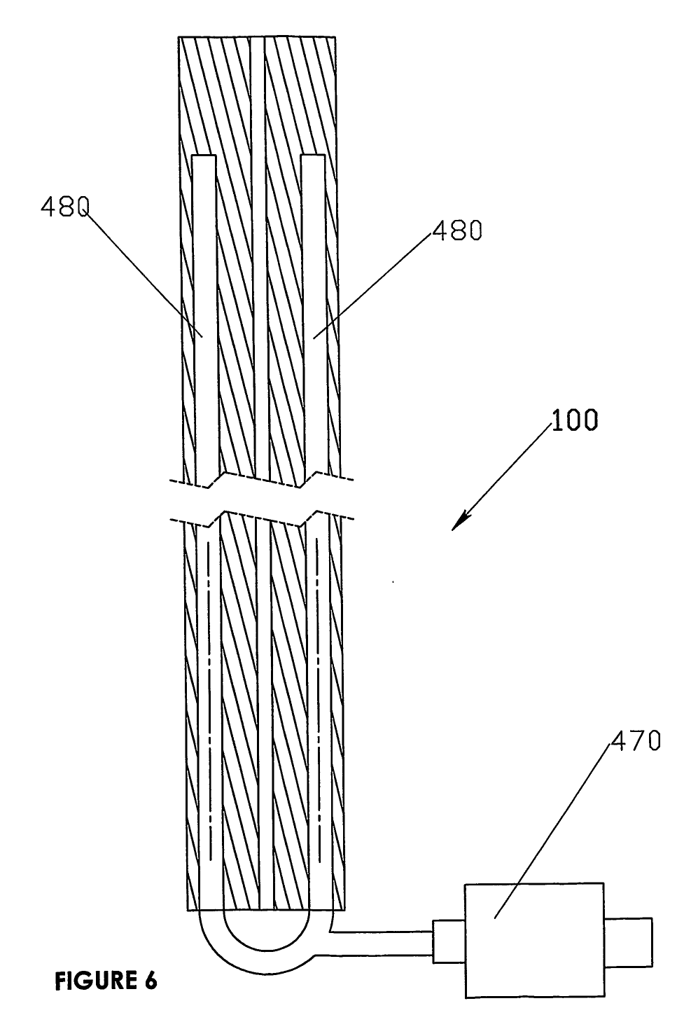

FIGS. 5A and 5B illustrate handle 400 in isometric and section view. In accordance with a preferred embodiment of the present invention, an operator steers head 200 by turning co-axially mounted steering controls (for example knobs) 440 and 441. Each knob is connected (via axis 460 and 450 respectively) to a pulley (461 and 460 respectively) around which loop cords 470 and 471 (respectively), which run from the respective knob, through trunk 100 and gimbals 700 to head 200. When the knob is turned it pulls one end of the cord and plays out the other end, thereby pulling head 200 back in the direction of the cord end that is being pulled. Cords 470 and 471 are at right angles to one another such that combined adjustment of controls 440 and 441 can turn head in any direction.

In a preferred embodiment of the present invention, a stiffening mechanism is provided to enable the operator to vary the stiffness of trunk 100. The reason for this is that at some junctures during insertion, for example when turning head 200 to negotiate curve in the patient's lumen, it is preferable for the trunk to be more pliant. At other junctures, such as when inserting head 200 further into the lumen, it is preferable for the trunk to be stiffer, thereby preventing it from buckling under the insertion force.

One embodiment of the stiffening mechanism is shown in FIGS. 5A and 5B. User control (for example, knob) 427 is attached to a pulley to which is attached one end of a cord 430 which runs through pulley 427 to steering axes 450 and 460. An operator can adjust the tension of cord 430, thereby adjusting the tension on axes 460 and 461, thereby adjusting the tension on cords 471 and 470, thereby compressing or relaxing trunk 200 and gimbals 700, to achieve greater or lesser stiffness.

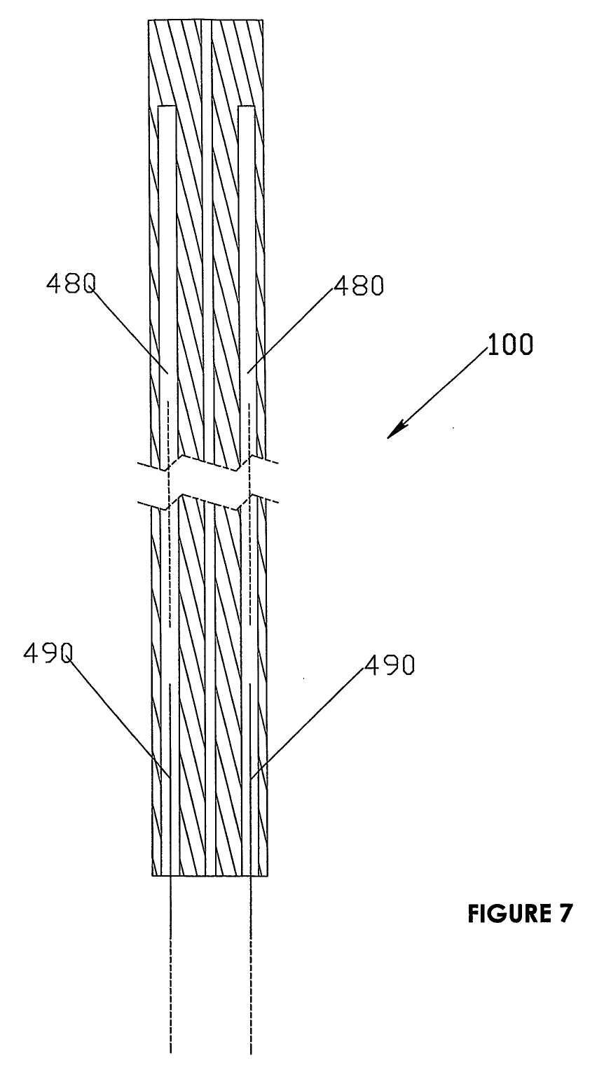

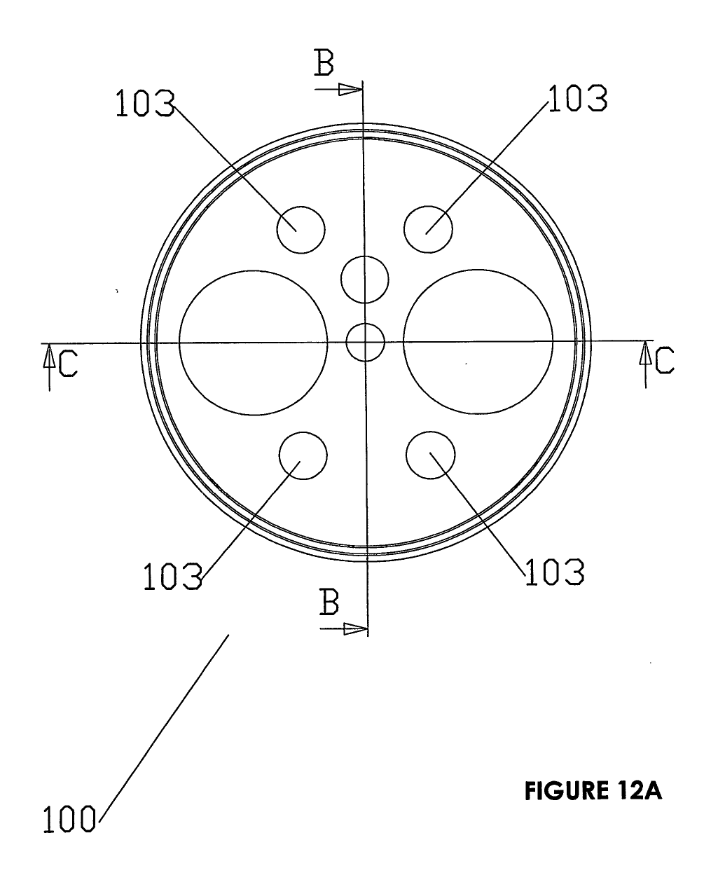

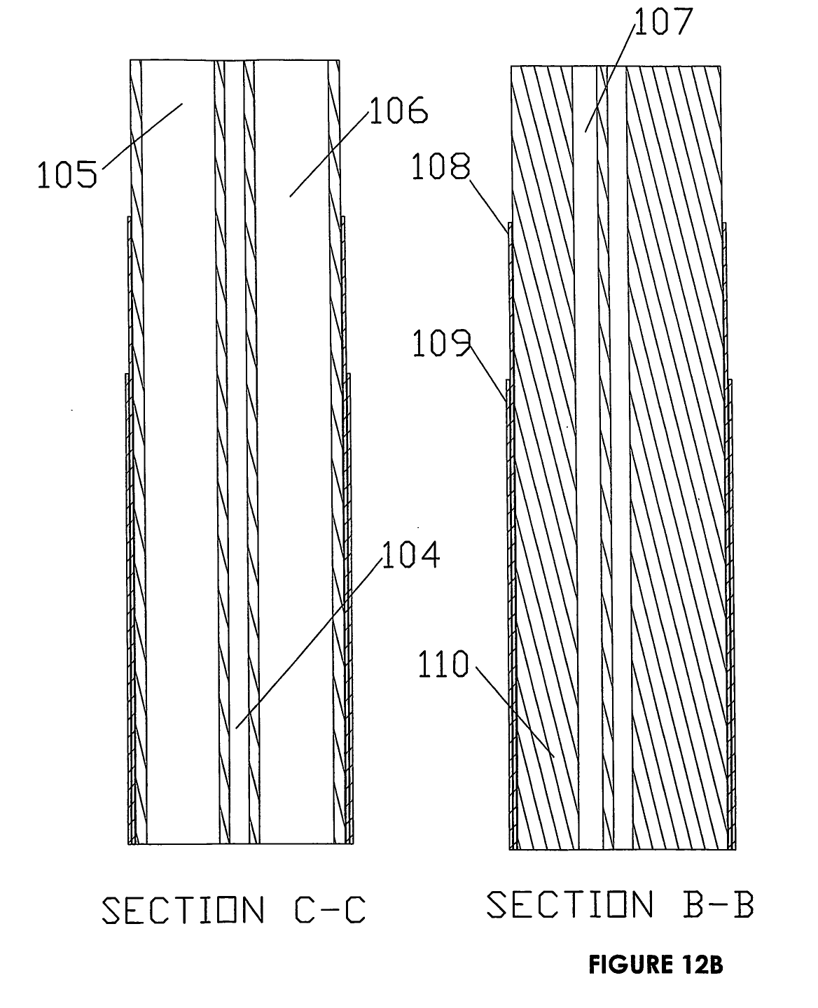

A stiffening mechanism for an alternative preferred embodiment of the present invention is shown in FIG. 6. Stiffening channels 480 run inside trunk 100 (see also FIG. 12A). Fluid pump 470 can be operated to insert or remove fluid, thereby controlling stiffness of trunk 100.

A stiffening mechanism for another alternative preferred embodiment of the present invention is shown in FIG. 7. Again, stiffening channels 480 (see also FIG. 12A) run inside trunk 100. This time the stiffening agent is a wire or rod 490 rather than fluid.

The present invention also provides several optional mechanisms for inserting the endoscope 1000 into the patient in addition to standard automated or manual drive mechanisms.

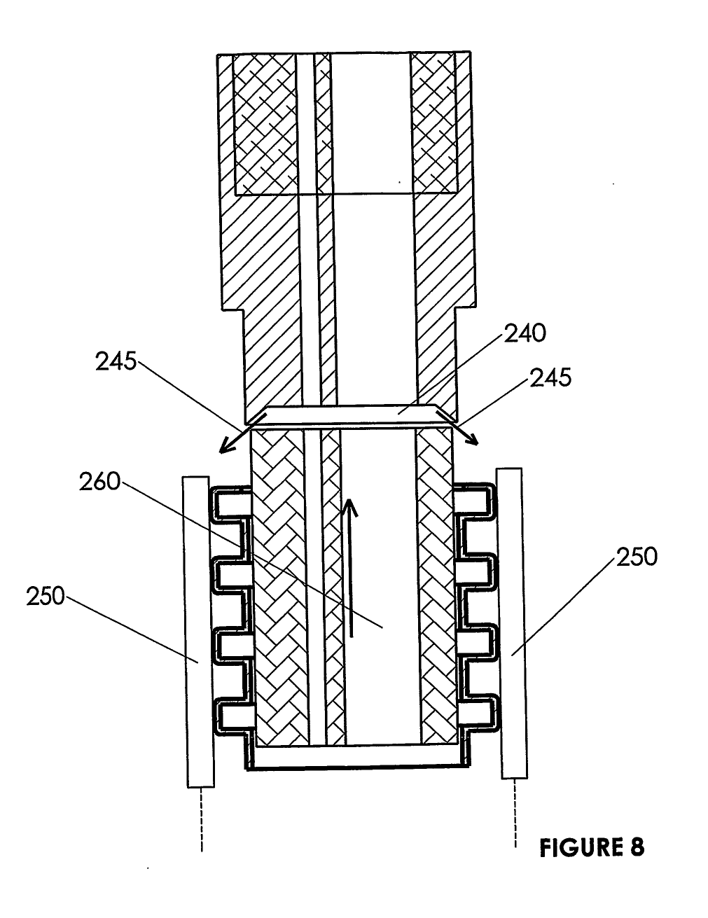

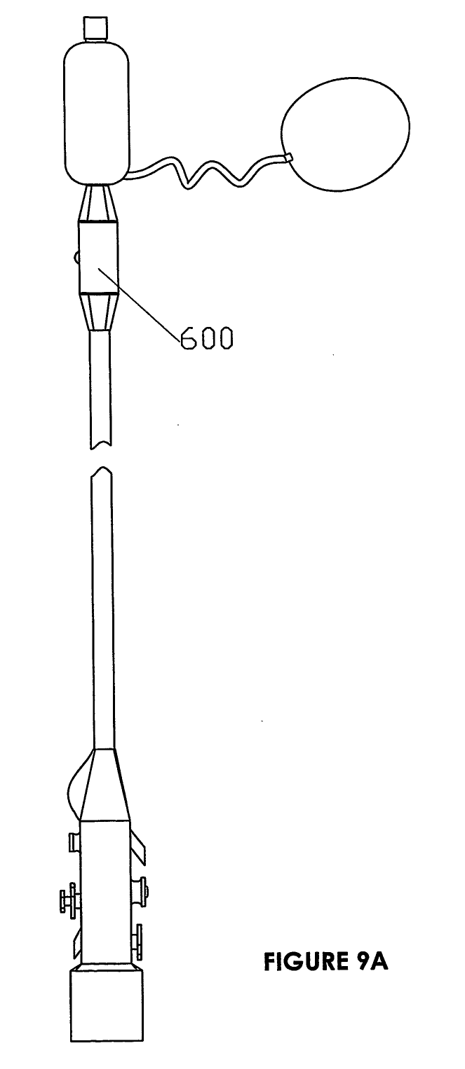

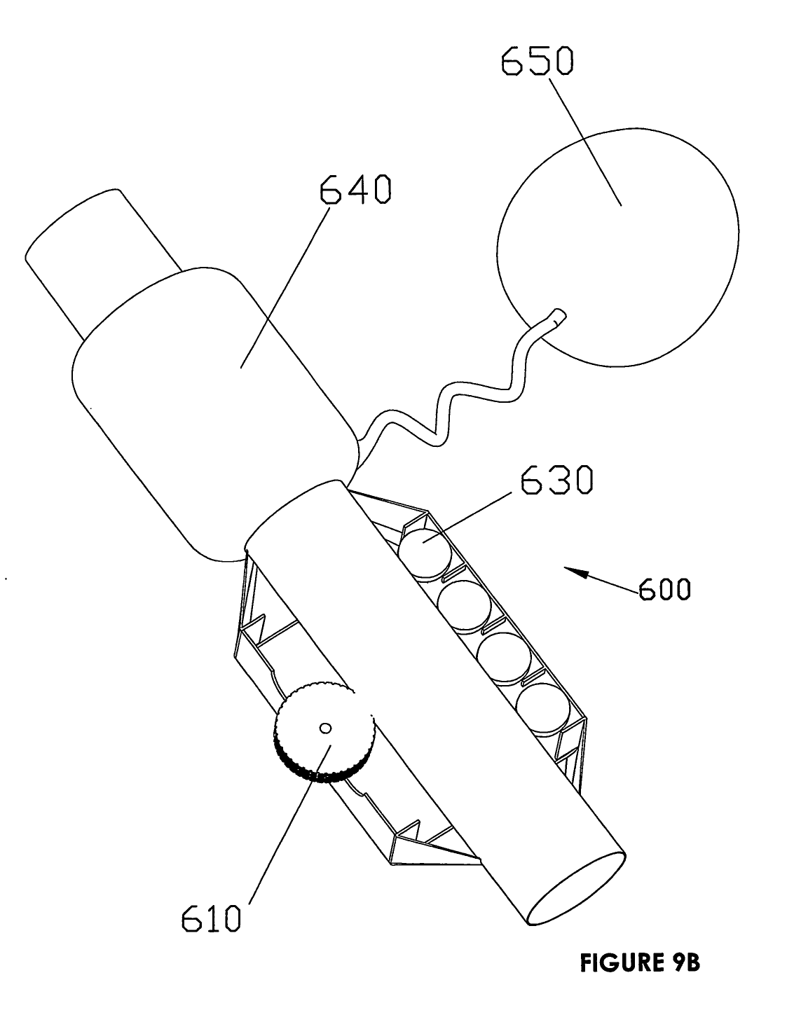

With reference to FIG. 8, in a preferred embodiment of the present invention, the insertion mechanism comprises a channel 260 through which a propelling fluid (for example, water) is pumped (illustrated by long arrow in FIG. 5), through trunk 100 and gimbals 700 to propulsion plate 240. There the fluid streams out the sides through exhaust 245 (shown with small arrows in FIG. 5) into the lumen, from where it is removed through exhaust channel 250, which is at least one of a plurality of tubes running back along on gimbals 700 and trunk 100. The force of the fluid on plate 240 drives the endoscope forward. If required, the insertion mechanism can be adapted to enable switching to removal mode, where the exhaust force is directed such that endoscope 1000 can be removed.

With reference to FIGS. 8A and 8B, another insertion (and in this case, also removal) mechanism 600 for an alternative preferred embodiment of the present invention features inflatable collar 640 which an operator inflates from bladder 650, to anchor endoscope 1000 in an orifice leading to the patient's lumen. For example, in the case of a gastrointestinal exam, collar 640 would anchor endoscope 1000 in the patient's anus. Once endoscope 1000 is anchored, the operator operates a mechanism that applies linear motion control (for example, a roller) 610 moving trunk 100. In the roller implementation shown, opposition is provided by opposition rollers 630. Depending on the direction of rotation of motion mechanism 610, trunk 100 is either inserted into, or retracted from, the patient.

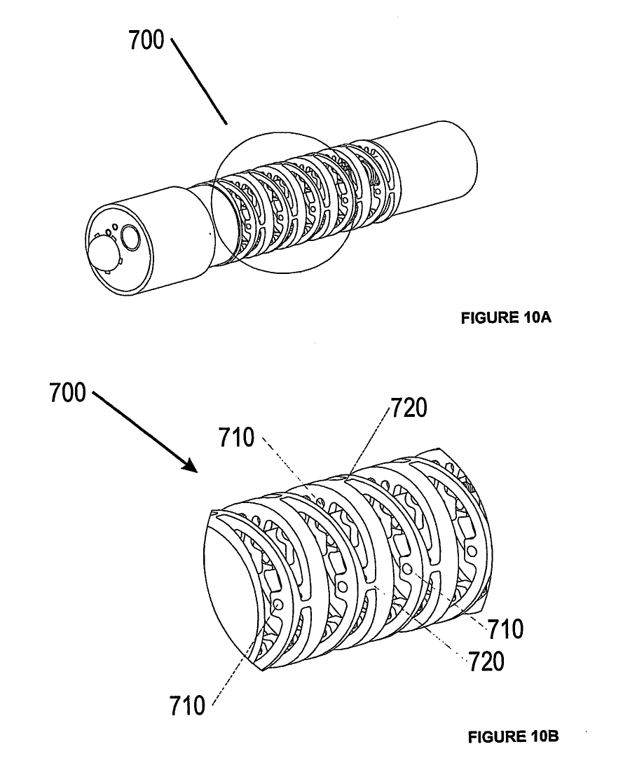

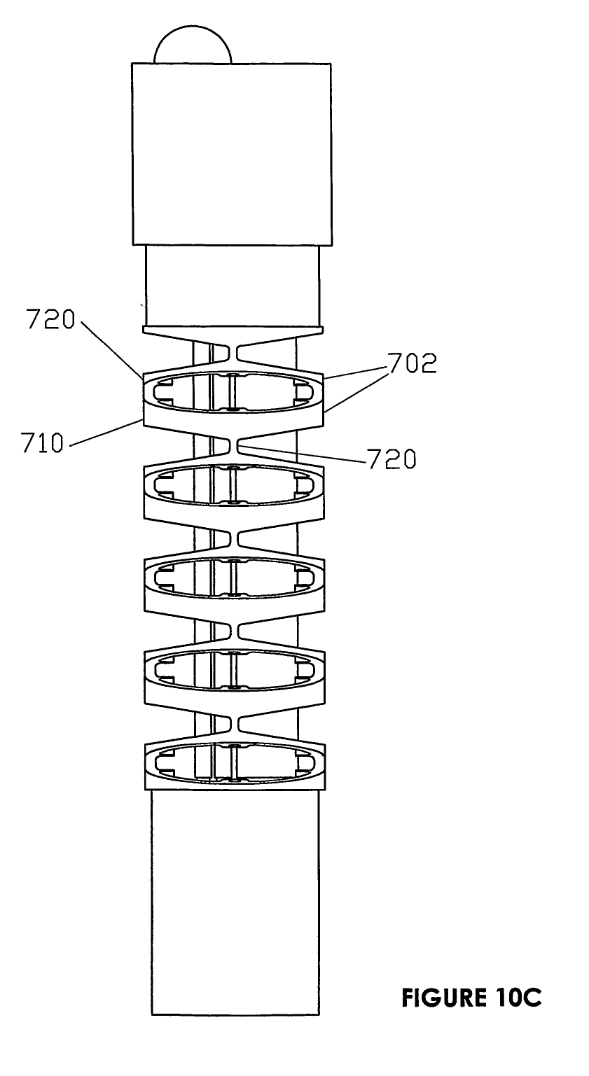

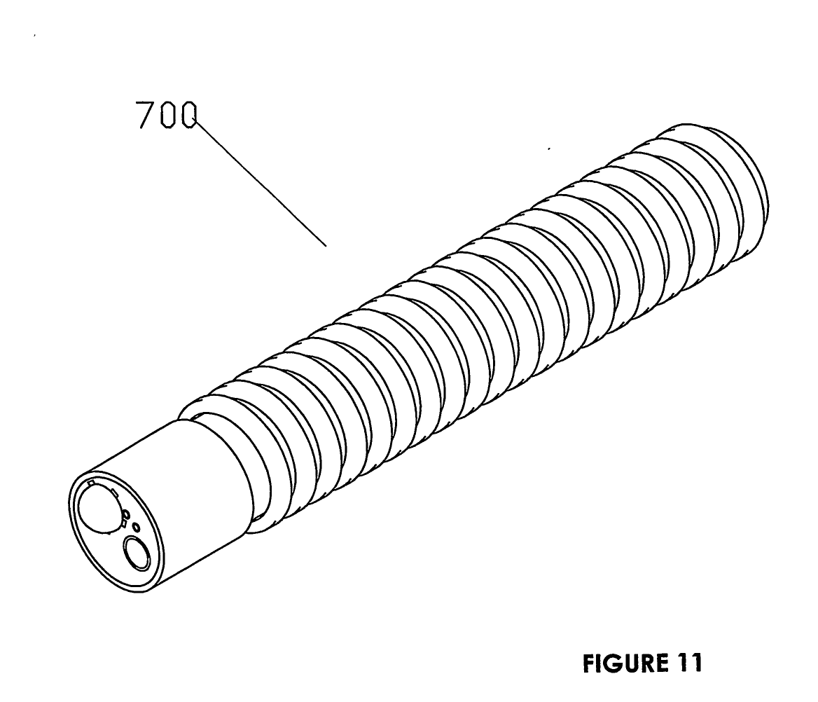

FIGS. 10A, 10B, and 10C illustrate a gimbals 700 for an endoscope in accordance with a preferred embodiment of the present invention.

Gimbals 700 comprises pairs of disks 702 that enable head 200 to pivot in any direction. Each disk 702 has internal spaces for the passage of materials, instruments, power and data, etc. In addition, each disk 702 has holes 710 spaced evenly around its perimeter (for example, at angles of 0, 90, 180 and 270 degrees from center). Each pair of opposing holes provides passage for the two sides of a steering cord 470 or 471. The cords terminate at the distal end of the device and are used to steer the device as was described earlier. Disks 702 are “hinged” to each other at two points 720. The members of the hinge pair are located opposite one another on the disk, each at the point where a hole 710 passes. The bridge pair on one face of a disk is oriented at 90 degrees to the bridge pair on the other face of the disk.

Operation of steering works as follows. When a steering cord 470 or 471 is retracted by control 440 or 441, it passes back through its series of holes, pulling back on head 200. The series of hinges 720 located along the path of the retracted cord act as blocks, preventing the adjoining disks from compressing, however the other series of hinges 720 (oriented at 90 degrees to the cord) function as hinges, allowing the adjoining disks to compress together, thereby causing gimbals 700 to turn in the direction of the retracted cord, thereby turning head 200.

Another gimbals 700 mechanism is provided in an alternative preferred embodiment of the present invention, as shown in FIG. 11. In this arrangement, simple ridged (“convoluted”) plastic tubing is used, the tubing comprising thinner-walled inner rings alternating with thicker-walled outer rings. When the string is retracted, the thinner inner rings compress along the string's path, thereby turning head 200.

The composition of trunk 100 is now described with reference to FIG. 12A, which is a top section view, and FIG. 12B, which comprises the two side section views indicated in FIG. 12A. Trunk 100 comprises a solid core 110 encompassing channels of various cross-sectional shapes running its length. The channels are intrinsic parts of core 110 and are created as part of the manufacturing process.

The use of the channels depends on the particular application. In most cases, the channels will be used as follows:
tensioning channels 480
steering cord channel 104
video link channel 105
instrument (“working”) channel 106
fluid evacuation channel 107

Trunk core 110 is encompassed in mesh 108, which prevents torsion of trunk 100. Mesh 108 is covered with sheath 109, which creates a smooth surface for reduced friction and protects mesh 108.

It should be clear that the description of the embodiments and attached Figures set forth in this specification serves only for a better understanding of the invention, without limiting its scope as covered by the following Claims.

It should also be clear that a person in the art, after reading the present specification could make adjustments or amendments to the attached Figures and above described embodiments that would still be covered by the following Claims.

1-29. (canceled)

30. An endoscope comprising:
(a) a flexible elongated solid body having:
(i) an elongated flexible trunk;
(ii) a distal head; and
(iii) a steerable portion supporting said distal head relative to said trunk, said steerable portion being operable from a proximal portion of said trunk to enable an operator to steer the head along twists and convolutions in the lumen,
the body including cavities provided for passing to or from the head materials, data connections, power connections, or instruments; and
(b) a mechanism for selectively altering mechanical properties of at least part of said trunk so as to enhance resistance of said at least part of said trunk to buckling under a force of insertion along the body lumen.

31. The endoscope of claim 30, wherein said mechanism is configured to selectively bias said at least part of said trunk towards a straightened state.

32. The endoscope of claim 31, wherein said mechanism includes at least one cable extending along at least part of said trunk, and a tension control mechanism for selectively increasing a tension in said at least one cable so as to alter said mechanical properties of said at least part of said trunk.

33. The endoscope of claim 32, wherein said at least one cable is part of a steering mechanism for steering said steerable portion.

34. The endoscope of claim 31, wherein said mechanism includes at least one elongated insert for insertion along an elongated cavity extending along said at least part of said trunk, said elongated insert being effective to alter said mechanical properties of said at least part of said trunk.

35. The endoscope of claim 31, wherein said mechanism includes at least one elongated inflatable cavity extending along said at least part of said trunk and configured such that, when filled with a fluid, said at least one elongated inflatable cavity alters said mechanical properties of said at least part of said trunk.

36. The endoscope of claim 35, wherein the fluid includes a liquid.

37. The endoscope of claim 35, wherein the fluid includes a gas.

38. The endoscope of claim 35, wherein said mechanism further includes a reservoir in fluid connection with said at least one elongated inflatable cavity for containing at least part of the fluid and for providing the fluid when needed.

39. The endoscope of claim 30, wherein the body is made of a plurality of separate parts, that are connectable.

40. The endoscope of claim 39, wherein the parts are provided with snap connectors for fast connection.

41. The endoscope of claim 30, further comprising an imaging arrangement deployed within said distal head, said imaging arrangement including:
(a) an image sensor array; and
(b) an optical arrangement for focusing an image of a viewed image onto said image sensor array,
wherein said image sensor array is deployed facing a direction non-parallel to a line of sight of said imaging arrangement, and wherein said optical arrangement is configured to direct a viewed image onto said image sensor array.

42. The endoscope of claim 41, wherein said image sensor array is deployed facing a direction substantially perpendicular to a line of sight of said imaging arrangement.

43. The endoscope of claim 41, wherein said image sensor array has a major dimension, and wherein said distal head has a central axis, said image sensor array being deployed with said major dimension substantially parallel to said central axis.

44. The endoscope of claim 41, wherein said optical arrangement includes a transparent protective cap deployed to ensure at least a given minimum spacing of objects from a lens of said optical arrangement.

45. An endoscope comprising:
(a) a flexible elongated solid body having:
(i) an elongated flexible trunk; and
(ii) a distal head; and
the body including cavities provided for passing to or from the head materials, data connections, power connections, or instruments; and
(b) an imaging arrangement deployed within said distal head, said imaging arrangement including:
(i) an image sensor array; and
(ii) an optical arrangement for focusing an image of a viewed image onto said image sensor array,
wherein said image sensor array is deployed facing a direction non-parallel to a line of sight of said imaging arrangement, and wherein said optical arrangement is configured to direct a viewed image onto said image sensor array.

46. The endoscope of claim 45, wherein said image sensor array is deployed facing a direction substantially perpendicular to a line of sight of said imaging arrangement.

47. The endoscope of claim 45, wherein said image sensor array has a major dimension, and wherein said distal head has a central axis, said image sensor array being deployed with said major dimension substantially parallel to said central axis.

48. The endoscope of claim 45, wherein said optical arrangement includes a transparent protective cap deployed to ensure at least a given minimum spacing of objects from a lens of said optical arrangement.
